(19) United States
(12) Patent Application Publication
(10) Pub. No.: US 2023/0302175 A1
(43) Pub. Date: Sep. 28, 2023

(54) STERILISATION APPARATUS FOR PRODUCING HYDROXYL RADICALS

(71) Applicant: Creo Medical Limited, Chepstow, Monmouthshire (GB)

(72) Inventors: Christopher Paul HANCOCK, Chepstow (GB); Jolyon DE FOSSARD, Newport (GB); Steven MORRIS, Bristol (GB)

(21) Appl. No.: 18/018,719

(22) PCT Filed: Jul. 15, 2021
(86) PCT No.: PCT/EP2021/069747

§ 371 Date: Jan. 30, 2023

(30) Foreign Application Priority Data
Aug. 5, 2020 (GB) 2012131.5

(51) Int. Cl. A61L 2/14; A61L 2/07; A61L 2/12

(52) U.S. Cl. CPC A61L 2/14; A61L 2/07; A61L 2/12; A61L 2101/34

(57) ABSTRACT

The invention relates to sterilisation systems suitable for clinical use, e.g. on the human body, medical apparatuses, or hospital bed spaces. In particular, the invention provides a sterilisation apparatus comprising: a sterilisation device having a hydroxyl radical generating region and an outlet for directing generated hydroxyl radicals out of the hydroxyl radical generating region towards a region to be sterilised; and a steam supply connected to deliver dry steam to the sterilisation device, wherein the sterilisation device is configured to generate a high impedance at the hydroxyl radical generating region when dry steam, radiofrequency (RF) and/or microwave frequency electromagnetic (EM) energy, and gas are delivered to the hydroxyl generating region, thereby to create a plasma of the gas which forms an ionisation discharge for generating hydroxyl radicals from the dry steam for delivery out of the hydroxyl generating region.

### FIELD OF THE INVENTION

The invention relates to sterilisation systems suitable for clinical use, e.g. on the human body, medical apparatuses, or hospital bed spaces. For example, the invention may provide a system that can be used to destroy or treat certain bacteria and/or viruses associated with the human or animal biological system and/or the surrounding environment. The invention relates to a sterilisation apparatus which generates hydroxyl radicals in an efficient way for sterilisation.

### BACKGROUND TO THE INVENTION

Bacteria are single-celled organisms that are found almost everywhere, exist in large numbers and are capable of dividing and multiplying rapidly. Most bacteria are harmless, but there are three harmful groups; namely: cocci, spirilla, and bacilla. The cocci bacteria are round cells, the spirilla bacteria are coil-shaped cells, and the bacilli bacteria are rod-shaped. The harmful bacteria cause diseases such as tetanus and typhoid.

Viruses can only live and multiply by taking over other cells, i.e. they cannot survive on their own. Viruses cause diseases such as colds, flu, mumps and AIDS. Viruses may be transferred through person-to-person contact, or through contact with region that is contaminated with respiratory droplets or other virus-carrying bodily fluids from an infected person.

Fungal spores and tiny organisms called protozoa can cause illness.

Sterilisation is an act or process that destroys or eliminates all form of life, especially micro-organisms. During the process of plasma sterilisation, active agents are produced. These active agents are high intensity ultraviolet photons and free radicals, which are atoms or assemblies of atoms with chemically unpaired electrons. An attractive feature of plasma sterilisation is that it is possible to achieve sterilisation at relatively low temperatures, such as body temperature. Plasma sterilisation also has the benefit that it is safe to the operator and the patient.

Plasma typically contains charged electrons and ions as well as chemically active species, such as ozone, nitrous oxides, and hydroxyl radicals. Hydroxyl radicals are far more effective at oxidizing pollutants in the air than ozone and are several times more germicidal and fungicidal than chlorine, which makes them a very interesting candidate for destroying bacteria or viruses and for performing effective decontamination of objects contained within enclosed spaces, e.g. objects or items associated with a hospital environment.

OH radicals held within a “macromolecule” of water (e.g. a droplet within a mist or fog) are stable for several seconds and they are 1000 times more effective than conventional disinfectants at comparable concentrations.

An article by Bai et al titled “Experimental studies on elimination of microbial contamination by hydroxyl radicals produced by strong ionisation discharge” (Plasma Science and Technology, vol. 10, no. 4, August 2008) considers the use of OH radicals produced by strong ionisation discharges to eliminate microbial contamination. In this study, the sterilisation effect on E. coli and B. subtilis is considered. The bacteria suspension with a concentration of 107 cfu/ml (cfu=colony forming unit) was prepared and a micropipette was used to transfer 10 μl of the bacteria in fluid form onto 12 mm×12 mm sterile stainless steel plates. The bacteria fluid was spread evenly on the plates and allowed to dry for 90 minutes. The plates were then put into a sterile glass dish and OH radicals with a constant concentration were sprayed onto the plates. The outcomes from this experimental study were:
1. OH radicals can be used to cause irreversible damage to cells and ultimately kill them;
2. The threshold potential for eliminating micro-organisms is ten thousandths of the disinfectants used at home or abroad;
3. The biochemical reaction with OH is a free radical reaction and the biochemical reaction time for eliminating micro-organisms is about 1 second, which meets the need for rapid elimination of microbial contamination, and the lethal time is about one thousandth of that for current domestic and international disinfectants;
4. The lethal density of OH is about one thousandths of the spray density for other disinfectants—this will be helpful for eliminating microbial contamination efficiently and rapidly in large spaces, e.g. bed-space areas; and
5. The OH mist or fog drops oxidize the bacteria into CO₂, H₂O and micro-inorganic salts. The remaining OH will also decompose into H₂O and O₂, thus this method will eliminate microbial contamination without pollution.

WO 2009/060214 discloses sterilisation apparatus arranged controllably to generate and emit hydroxyl radicals. The apparatus includes an sterilisation device which receives RF or microwave energy, gas and water mist in a hydroxyl radical generating region. The impedance at the hydroxyl radical generating region is controlled to be high to promote creation of an ionisation discharge which in turn generates hydroxyl radicals when water mist is present. The sterilisation device may be a coaxial assembly or waveguide. A dynamic tuning mechanism e.g. integrated in the sterilisation device may control the impedance at the hydroxyl radical generating region. The delivery means for the mist, gas and/or energy can be integrated with each other.

WO 2019/175063 discloses a sterilisation apparatus that uses thermal or non-thermal plasma to sterilise or disinfect surgical scoping devices. In one example, a plasma generating region is formed at a distal end of a coaxial transmission line, which convey RF or microwave energy to strike and sustain the plasma. A gas passageway is formed around an outer surface of the coaxial transmission line. The gas passageway is in fluid communication with the plasma generating region through notches in a cylindrical electrode mounted on a distal end of the coaxial transmission line. In some examples, water through a passageway formed within the inner conductor of the coaxial transmission line, from where it is sprayed on to the surface of an object before the plasma passes over it.

To optimise the generation of hydroxyl radicals, there is a need to introduce more water into close proximity with the most reactive part of the plasma discharge. If water is introduced to a sterilisation device too far away from the plasma discharge, there is a risk that the ionised gas no longer has enough energy to promote the required chemical reaction. Water may also build up inside the plasma sterilisation device, which can inhibit plasma generation, resulting in fewer or no hydroxyl radicals being generated.

### SUMMARY OF THE INVENTION

At its most general, the invention provides a sterilisation apparatus having an improved hydroxyl (OH) radical generation efficiency by generating OH radicals from steam.

According to a first aspect of the invention, there is provided a sterilisation apparatus comprising: a sterilisation device having a hydroxyl radical generating region and an outlet for directing generated hydroxyl radicals out of the hydroxyl radical generating region towards a region to be sterilised; and a steam supply connected to deliver dry steam to the sterilisation device, wherein the sterilisation device is configured to generate a high impedance at the hydroxyl radical generating region when dry steam, radiofrequency (RF) and/or microwave frequency electromagnetic (EM) energy, and gas are delivered to the hydroxyl generating region, thereby to create a plasma of the gas which forms an ionisation discharge for generating hydroxyl radicals from the dry steam for delivery out of the hydroxyl generating region. In some embodiments, the sterilisation apparatus may additionally comprise a gas supply connected to supply gas to the sterilisation device; and a generator configured to generate RF and/or microwave frequency EM energy and connected to supply the RF and/or microwave EM energy to the sterilisation device.

By providing a sterilisation apparatus in this way, which is arranged to generate hydroxyl radicals from dry steam rather than a water mist as in known arrangements, the present invention improves the efficiency of hydroxyl radical production for sterilisation. In particular, the use of dry steam helps to reduce the build-up of water within the sterilisation device at the hydroxyl radical generating region which would otherwise inhibit plasma generation. For example, the build-up of water within the hydroxyl radical generating region may shunt an RF pulse (used to strike a thermal or non-thermal plasma) to ground, preventing a plasma being struck.

Furthermore, even if a plasma is struck in such conditions, water within the hydroxyl generating region represents a well-matched load for microwave energy, and so the microwave energy is dissipated to the water rather than adding energy to the plasma. The energy dissipated to the water may eventually heat the water to provide a mist or vapour, but at the cost of energy which could be delivered to the plasma to generate hydroxyl radicals. By overcoming these problems, the present invention provides a sterilisation apparatus having an improved hydroxyl (OH) radical generation efficiency.

Herein, the term “dry steam” is used to refer to gaseous water which contains substantially no liquid component (for example, less than 5% or less than 1% by volume), for example in the form of condensed water droplets, and consists substantially entirely of gas. In contrast, the term “wet steam” as used herein may refer to steam comprising of a combination of liquid, for example in the form of condensed water droplets and gas.

Preferably, the steam supply may comprise a water mist generator and a heating element, wherein the heating element is arranged to receive water mist from the water mist generator and heat the mist to produce dry steam. For example, the water mist generator may comprise an ultrasound transducer positioned in a bath of water to produce water mist. In some examples, gas may be delivered to the mist generator from a gas supply in order to entrain mist in a flow of the gas. For example, gas may be bubbled through a water bath in order to produce a stream of water droplets entrained within a flow of the gas. The mist and gas mixture may be heated in order to produce a mixture of gas and dry steam which can be delivered simultaneously to the sterilisation device. As used herein, the term “water mist” may be used to describe a suspension of condensed water droplets in air or another gas (e.g. gas from a gas supply, such as Argon gas).

Advantageously, the heating element may be positioned in a mist flow path between the water mist generator and the sterilisation device. That is, the heating element may be physically positioned in a mist flow path in order to contact water mist flowing along the mist flow path in order to heat the water mist to produce dry steam.

Preferably, at least a portion of a mist flow path between the water mist generator and the sterilisation device may be provided by a pipe, wherein the heating element is arranged to heat the pipe either directly or indirectly to generate dry steam. For example, the pipe may be immersed in a reservoir of a liquid having a high specific heat capacity, and wherein the heating element is arranged to heat the liquid within the reservoir. For example, the heating element may be in contact with the liquid within the reservoir, such that the liquid can heat the pipe, thereby heating the water mist flowing therethrough to produce dry steam. A high specific heat capacity may be a specific heat capacity of at least 1.5 J/g° C. For example, the liquid may be an oil, such as silicone oil or the like, or a petroleum hydrocarbon based oil. The heating element may be arranged to heat the liquid with the reservoir to a temperature of at least 140° C., for example 180° C., or 200° C.

Advantageously, the steam supply may comprise a steam generator and a steam dryer, wherein the steam dryer is arranged to receive wet steam from the steam generator and to remove water droplets from the wet steam to produce dry steam. The steam generator may comprise a resistive heating element for heating water to produce wet steam, for example. In one embodiment, the steam generator may comprise a boiler into which water is introduced at a constant rate to be heated by a heating element, and so produces a substantially constant output of wet steam to be passed to a steam dryer. For example, water may be introduced to the steam generator at a rate of 4 litres per minute.

In some examples, the steam dryer may comprise a steam trap, labyrinth, gauze or the like for removing water droplets from wet steam supplied by the steam generator. For example, the steam dryer may comprise a thermally insulated tortuous path through which the wet steam flows, removing water droplets from the wet steam to produce dry steam.

Alternatively, the steam dryer may comprise a heating element, such as a resistive heating element, such that the steam dryer is configured to produce superheated steam, i.e. dry steam which is at a temperature of greater than 100° C. For example, the superheated steam may be heated to a temperature of at least 100° C., such as at least 110° C., or at least 130° C.

To superheat the wet steam, the heating element may be arranged to heat a portion of a steam flow path between the steam generator and the sterilisation device, either directly or indirectly. For example, the steam dryer may comprise a heating element which is positioned in the steam flow path between the steam generator and the sterilisation device. That is, the heating element may be physically positioned in the steam flow path in order to contact wet steam in order to deliver heat thereto for producing dry steam. In some examples, the steam dry may comprise a first heating element (or other heat source which is used to initiate dry steam production, and a second heating element which may is used to maintain dry steam production, wherein the first heating element may provide more thermal energy per second than the second heating element.

In one embodiment, the steam flow path may comprise a pipe, such as a metal pipe, which may be heated by the heating element either directly or indirectly in order to deliver heat to wet steam passing therethrough to produce dry steam. In a preferred embodiment, the pipe may be immersed in a reservoir of a liquid having a high specific heat capacity, the heating element being arranged to heat the liquid within the reservoir. A high specific heat capacity may be a specific heat capacity of at least 1.5 J/g° C. For example, the liquid may be an oil, such as silicone oil or the like, or a petroleum hydrocarbon based oil. The heating element may be arranged to heat the liquid with the reservoir to a temperature of at least 140° C., for example 180° C., or 200° C.

A steam dryer provided in this way (which may be referred to herein as a superheater) enables the provision of a suitable amount of dry steam to the sterilisation apparatus. A constant flow rate may be easily achieve as the high specific heat capacity liquid can be readily maintained at a constant temperature while providing enough thermal energy to wet steam to enable production of dry steam. For example, the superheater may be used to provide dry steam at a rate between 2 and 6 litres per minute, preferably around 4 litres per minute or less. By providing a reservoir of a liquid having a high specific heat capacity, the steam may also be superheated to a consistent temperature, which may be easily maintained by heating the liquid using the heating element.

Preferably the sterilisation apparatus further comprises a gas supply connect to supply gas to the sterilisation device, wherein the gas supply is a source of heated gas. For example, the gas supply may provide gas at a rate of at least 10 litres per minute. Steam and gas may mix in the hydroxyl radical generating region, or before reaching the hydroxyl radical generating region, and so providing heated gas can reduce or eliminate steam condensing in the hydroxyl generating region. For example, the gas supply may comprise a gas heating element, which may be a resistive heating element for example. Preferably, the gas supply may be configured to heat the gas to a temperature of at least 100° C., such as at least 110° C., or at least 130° C.

Preferably, the steam supply may comprise a diverter valve in order to allow an operator of the sterilisation apparatus to easily adjust the amount of dry steam which is delivered to the sterilisation device.

In one example, the gas heating element may be positioned in a gas flow path between the gas supply and the sterilisation device. That is, the gas heating element may be physically positioned in the gas flow path to be in contact with the gas and deliver heat thereto for producing heated steam.

Alternatively, at least a portion of a gas flow path between the gas supply and the sterilisation device may be provided by a pipe, wherein the heating element may be arranged to heat the pipe, either directly or indirectly. In a preferred embodiment, the pipe may be immersed in a reservoir of a liquid having a high specific heat capacity, and the gas heating element may be arranged to heat the liquid within the reservoir, thereby delivering thermal energy to the pipe in order to deliver heat to gas passing through the pipe to produce heated gas. A high specific heat capacity may be a specific heat capacity of at least 1.5 J/g° C. For example, the liquid may be an oil, such as silicone oil or the like, or a petroleum hydrocarbon based oil. The heating element may be arranged to heat the liquid within the reservoir to a temperature of at least 140° C., for example 180° C., or 200° C. Advantageously, the sterilisation apparatus may further comprise a heater for heating the sterilisation device. In this way, cooling of the dry steam and condensing of water within the sterilisation device may be minimised or eliminated to ensure efficient generation of hydroxyl radicals. For example, the sterilisation apparatus may be provided with a resistive heating element, which may at least partially surround or enclose the hydroxyl generating region, e.g. a tungsten filament heating element.

Preferably, the sterilisation apparatus may further comprise a mixer arranged to receive dry steam from the steam supply and gas from the gas supply, and deliver a dry steam and gas mixture to the sterilisation device. For example, the mixer may preferably be a venturi mixer or the like. In some embodiments, the mixer may be heated to minimise heat losses and to help ensure that the steam does not condense in mixer or in the sterilisation device. Preferably, the dry steam and gas mixture may comprise at least 25% dry steam by volume.

Preferably the mixer may comprise at least one flow regulating valve which allows the proportion of dry steam and/or gas in the dry steam and gas mixture to be adjusted by an operator of the sterilisation apparatus.

Preferably the gas supply is a supply of any suitably inert gas for formation of a non-thermal or thermal plasma, for example argon, helium, nitrogen, carbon dioxide or a combination thereof. For example, the gas supply may be a pressurised supply of such a gas.

Advantageously, the sterilisation apparatus may also include an enclosure as well as a sterilisation device. The enclosure may be provided to define a region to be sterilised or may be used to confine hydroxyl radicals to an area proximate to an object to be sterilised. In this way, the hydroxyl radicals may be introduced into an enclosed environment that can be filled with a concentration of radicals suitable for filling bacteria or contaminants that exist inside the enclosure. For example, an enclosure may be used for personal protective equipment (PPE) sterilization (e.g. by placing items of PPE within the enclosure), or sterilisation on board trains and ambulances or the like, or to define an area within a building for localised sterilisation.

Herein, the term “inner” means radially closer to the centre (e.g. axis) of the coaxial cable, probe tip, and/or sterilisation device. The term “outer” means radially further from the centre (axis) of the coaxial cable, probe tip, and/or sterilisation device.

The term “conductive” is used here to mean electrically conductive, unless the context dictates otherwise.

Herein, the terms “proximal” and “distal” refers to the ends of the sterilisation device. In use, the proximal end is closer to a generator for providing the RF and/or microwave energy, whereas the distal end is further from the generator.

In this specification “microwave” may be used broadly to indicate a frequency range of 400 MHz to 100 GHz, but preferably in the range 1 GHz to 60 GHz. Specific frequencies that have been considered are: 915 MHz, 2.45 GHz, 3.3 GHz, 5.8 GHz, 10 GHz, 14.5 GHz, and 25 GHz. In contrast, this specification uses “radiofrequency” or “RF” to indicate a frequency range that is at least three orders of magnitude lower, e.g. up to 300 MHz, preferably 10 kHz to 1 MHz, and most preferably 400 kHz. The microwave frequency may be adjusted to enable the microwave energy delivered to be optimised. For example, a probe tip may be designed to operate at a certain frequency (e.g. 900 MHz), but in use the most efficient frequency may be different (e.g. 866 MHz).

The term “electrosurgical” is used in relation to an instrument, apparatus, or tool which is used during surgery and which utilises radiofrequency and/or microwave frequency electromagnetic (EM) energy.

### BRIEF DESCRIPTION OF THE DRAWINGS

Features of the invention are now explained in the detailed description of examples of the invention given below with reference to the accompanying drawings, in which:

FIG. 1 is a schematic diagram of a sterilisation apparatus according to an embodiment of the present invention;

FIG. 2 is a cross-sectional view of a sterilisation device which may be used in embodiments of the present invention;

FIG. 3 shows a schematic diagram of a gas supply and a steam supply which may be used in embodiments of the present invention;

FIG. 4 shows a cross-section view of a first heating device which may be used in embodiments of the present invention;

FIG. 5 shows a cross-section view of a second heating device which may be used in embodiments of the present invention; and

FIG. 6 shows a cross section view of a mixer which may be used in embodiments of the present invention.

### DETAILED DESCRIPTION; FURTHER OPTIONS AND PREFERENCES

This invention relates to an apparatus for performing sterilisation using hydroxyl radicals that are generated by creating a plasma in the presence of steam.

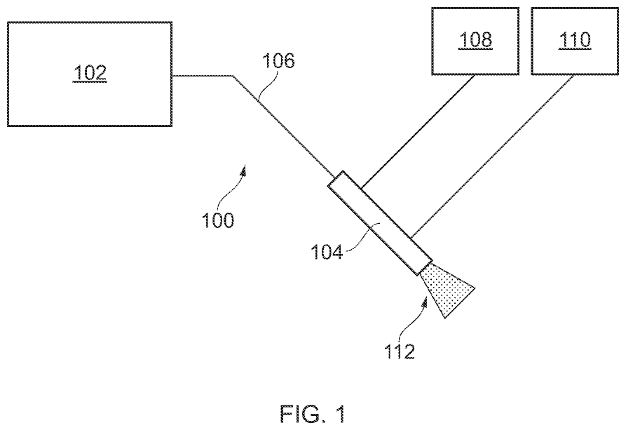

FIG. 1 is a schematic diagram of a sterilisation apparatus 100 which is an embodiment of the present invention.

The apparatus 100 is capable of generating hydroxyl (OH) radicals in order to sterilise a surface or an area. For example, the apparatus 100 may be used to sterilise medical apparatuses or hospital bed spaces.

The apparatus 100 comprises a generator 102 which is able to controllably deliver radiofrequency (RF) and/or microwave electromagnetic (EM) energy to a sterilisation device 104, which may be referred to herein as a sterilisation device, which is preferably a handheld unit.

The generator 102 may be of the type disclosed in WO 2012/076844, for example. The sterilisation device 104 may be an applicator or sterilisation device of the type disclosed in GB 2006384.8, GB 2006383.0, or WO 2009/060214, for example.

The generator 102 is connected to the sterilisation device 104 by a coaxial cable 106. The coaxial cable 106 comprises an inner conductor, an outer conductor and a dielectric material separating the inner conductor from the outer conductor. The coaxial cable 106 may couple energy into the sterilisation device 104 through a QMA connector or the like. In some examples, the generator 102 may be arranged to monitor reflected signals (i.e. reflected power) received back from the sterilisation device 104 in order to determine an appropriate signal to be conveyed to the sterilisation device 104. The radiofrequency and/or microwave energy is utilised at the sterilisation device 104 in order to strike and sustain a thermal or non-thermal plasma in order to generate hydroxyl radicals in a manner which is explained in more detail below.

In the apparatus shown in FIG. 1, a single generator 102 is arranged to supply RF and/or microwave frequency EM energy. However, in some embodiments of the present invention, the apparatus may comprise an RF EM energy generator and a microwave energy EM generator as individual components, which are each connected to the sterilisation device 104 by a respective coaxial cable.

The apparatus 100 further comprises a steam supply 108, which is arranged to deliver dry steam to the sterilisation device 104. Description of the steam supply 108 is given in more detail below. The steam supply 108 may include a pump or other fluid driving unit to cause generated steam to flow towards the sterilisation device 104. The dry steam is supplied to the sterilisation device 104 in order to generate hydroxyl radicals by a process which will be explained in more detail below. By using dry steam in this way the apparatus 100 can be used to sterilise surfaces or objects without the use of any cleaning chemicals, reducing costs associated with sterilisation and allowing sterilisation to be performed when cleaning chemicals are in short supply. The use of hydroxyl radicals for sterilisation also ensures that there are no harmful by-products. Furthermore, the use of dry steam to generate hydroxyl radicals helps to prevent the build-up of water within the sterilisation device 104 which would inhibit plasma and hydroxyl radical generation.

A gas supply 110 is connected to the sterilisation device 104 to supply gas for forming a plasma which is used to generate hydroxyl radicals in a manner which will be explained below. The gas supply 110 may be a pressurised supply of any suitably inert gas for formation of a non-thermal or thermal plasma, for example argon, helium, nitrogen, carbon dioxide or a combination thereof. The gas supply 110 may be configured to allow adjustment of the flow rate of gas which is delivered to the sterilisation device 104. The gas supply can supply between 1.5 and 15 litres of gas per minute, for example. In some embodiments, the gas supply 110 may be configured to supply heated gas to the sterilisation device 104, as explained in more detail below.

The gas supply 110 and steam supply 108 may be connected to the sterilisation device 104 by a common feed line. That is, outputs from the gas supply 110 and steam supply 108 may be combined before they reach the sterilisation device 104. For example, the sterilisation apparatus 100 may comprise a mixer arranged to receive steam from the steam supply 108 and gas from the gas supply 110, and deliver a steam and gas mixture to the sterilisation device 104. The combined gas/steam stream may be delivered into the sterilisation device 104 through a single fluid conduit. Alternatively, the gas supply 110 and the steam supply 108 may provide separate streams for delivery of steam and gas. The separate streams may be provided within a combined conduit. For example, a conduit for conveying gas to the sterilisation device 104 may comprise a T-junction to allow steam to be fed into the conduit. Alternatively, as shown in FIG. 1, the gas supply 110 and the steam supply 108 may be separately connected to the sterilisation device 104.

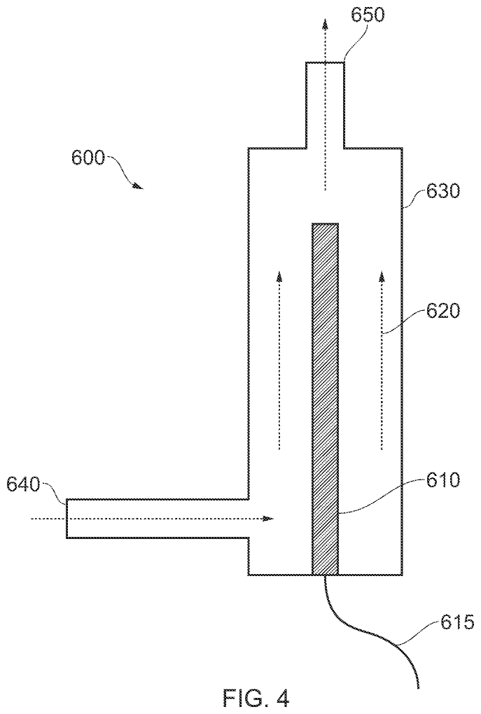

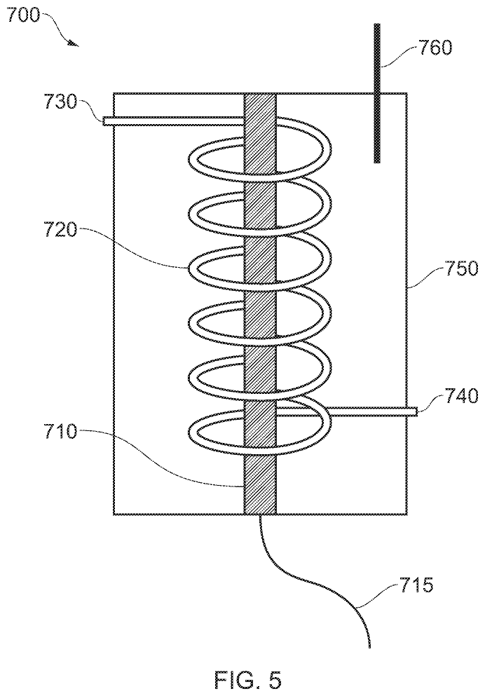

In another example, the gas supply 110 may be connected to the steam supply 108 in order to deliver a combined gas and dry steam stream to the sterilisation device. For example, the steam supply 108 may comprise a water bath through which gas from the gas supply 110 is passed (e.g. in the manner of a bubbler) in order to entrain water mist in the gas flow. The combined gas and water mist stream may be heated (e.g. using a heating device as shown in FIG. 4 or 5) in order to produce a flow of gas and dry steam for delivery to the sterilisation device. Alternatively, the steam supply 108 may be configured to generate a flow of water mist (e.g. using an ultrasound transducer or the like) to which gas may be introduced. The water mist may be heated with the gas, or the gas and water mist may be heated independently in order to produce a dry steam.

In some embodiments of the invention it is envisaged that the generator 102 (or multiple generators where present), the steam supply 108 and the gas supply 110 may each be portable, and the sterilisation device 104 may be a handheld sterilisation device such that the present invention provides an effective sterilisation apparatus which is easily transportable by a user. For example, the generator 102 may be powered by a battery or the like.

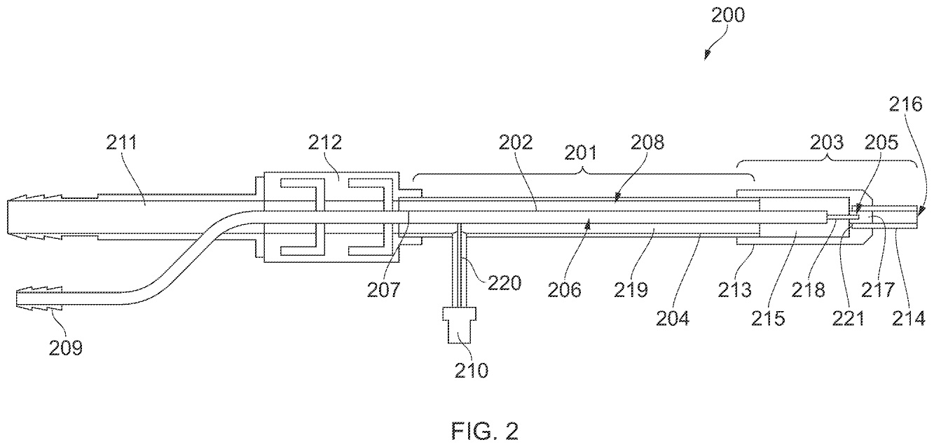

FIG. 2 shows a cross-sectional view of a sterilisation device 200 that may be used in embodiment of the invention, for example the sterilisation apparatus 100 shown in FIG. 1. Although not shown in FIG. 2, the sterilisation device 200 may be contained within a generally elongate housing which allows a user to easily pass the sterilisation device 200 over a surface or object for sterilisation. In some arrangements the sterilisation device 200 may be handheld unit to facilitate manual control.

The sterilisation device 200 comprises an energy delivery structure in the form of a coaxial transmission line 201 for conveying radiofrequency (RF) and/or microwave frequency electromagnetic (EM) energy. The coaxial transmission line 201 comprises an inner conductor 202 and an outer conductor 204 spaced away from the inner conductor 202 to define an annular region 219 therebetween. For example the inner conductor 202 may have an outer diameter of 3 mm and the outer conductor 204 may have an inner diameter of 7 mm to provide a suitable spacing. The spacing between the inner conductor 202 and the outer conductor 204 may be maintained by radially extending spacers (not shown) which are positioned in the gap, for example the spacers may be spokes or spoked discs made of PTFE.

A distal tip 203 is mounted at a distal end of the coaxial transmission line 201. The distal tip 203 comprises a cylindrical cap 213, which is an electrically conductive structure electrically connected to the outer conductor 204 of the coaxial transmission line 201. In this example, the cylindrical cap 213 comprises a proximal region that overlies and contacts an outer surface of the outer conductor 204. The cylindrical cap 213 defines an internal volume 215. The inner conductor 202 of the coaxial transmission line 201 protrudes beyond a distal end of the outer conductor 204 into the internal volume. The cylindrical cap 215 has an outlet aperture 217 in its distal end. The internal volume 215 is in fluid communication with an external environment through the outlet aperture 217. In this example, an insulating pipe 214 (e.g. formed from quartz or the like) is mounted in the outlet aperture, such that the internal volume 215 communicates with the external environment through a passageway formed by the insulating pipe 214.

The inner conductor 202 is hollow to form a steam conduit 206 for conveying dry steam along the coaxial transmission line 201 from a proximal inlet 207 to the internal volume 215 within the distal tip 203. A flow of dry steam is fed into the proximal inlet 207 via a steam input pipe 209, such that the flow of dry steam is parallel with the longitudinal axis of the inner conductor 202. This arrangement allows a high steam flow rate due to the lack of curves or bends in the steam conduit 206. The steam input pipe 209 receives steam from a steam supply, as described above with respect to FIG. 1.

The annular region 219 between the inner conductor 202 and the outer conductor 204 forms a fluid conduit 208 for conveying gas to the internal volume 215. Gas is delivered to the fluid conduit 208 through a gas input pipe 211, which is connected to a gas supply as described above with respect to FIG. 1.

In a preferred example, the sterilisation device 200 may be operated by conveying a mixture of gas and dry steam through the fluid conduit 208. In such operation, no steam is required to be delivered through the steam conduit 206, though steam may be simultaneously supplied through the steam conduit 206 if necessary.

RF and/or microwave energy is supplied to the coaxial transmission line 201 via a transverse coaxial feed 220. The transverse coaxial feed 220 couples the RF and/or microwave energy into the coaxial transmission line 201 at a location positioned towards a proximal end of the coaxial transmission line 201. To enable RF energy to be conveyed by the coaxial transmission line 201, the coaxial transmission line 201 has an open circuit condition at its proximal end (i.e. the inner conductor 202 and outer conductor 204 remain isolated from each other). To ensure efficient coupling of the microwave energy into this coaxial transmission line 201, the transverse coaxial feed is preferably positioned away from the proximal end of the coaxial transmission line by a distance equal to one or more half wavelengths of the microwave energy when propagating on the coaxial transmission line 201.

The transverse coaxial feed 220 has a connector 210 that is detachably connectable to a coaxial cable that conveys RF and/or microwave energy from a generator, as described above with respect to FIG. 1. For example, the connector 210 may comprise a QMA connector, a SMA connector or the like.

In order to prevent microwave energy from flowing past the proximal end of the coaxial transmission line 201, a choke 212 is connected at the proximal end of the coaxial transmission line 201. In this example a double choke arrangement is used. The choke 212 is provided with a longitudinal passage therethrough to admit the steam input pipe 209 and to provide fluid communication between the gas input pipe 211 and annular region 219.

As described above, the cylindrical cap 213 is open at its distal end, with an insulating pipe 214 positioned within the outlet aperture 217. A proximal region of the insulating pipe 214 defines a plasma generating zone 205, or hydroxyl radical generating region. A first electrode 218 that is electrically connected to the inner conductor 202 extends into the plasma generating zone 205. In this example, the first electrode 218 is a hollow conductive rod that protrudes from a distal end of the inner conductor 202. The rod has a smaller outer diameter than the outer diameter of the inner conductor 202. The steam conduit 206 may be in fluid communication with a longitudinal passage through the first electrode 218. The longitudinal passage may have a smaller diameter than that water conduit 206 so that the speed of steam flow in the longitudinal passage is increased relative to the steam conduit 206, i.e. the dry steam accelerates towards the plasma generating region 205.

A second electrode 221 is provided by one more radially protruding conductive tabs formed on the side surfaces of the outlet aperture 217 at a proximal end of the insulating pipe 214. Energy supplied to the coaxial transmission line 201 may thus cause a high voltage condition and a high impedance to exist between the first electrode 218 and second electrode 221 within the plasma generating zone 205, such that a plasma can be struck from gas supplied through the fluid conduit 208. The plasma may be struck by a pulse of RF energy and then sustained by a subsequent microwave EM pulse or pulses, forming an ionisation discharge for generating hydroxyl radicals from the dry steam delivered to the plasma generating zone 205. In other embodiments either RF or microwave EM energy alone may be used to strike and/or to sustain the plasma.

A benefit of forming the second electrode as discrete tabs is that it has less effect on the impedance in the cylindrical cap, and hence assists in efficient coupling of energy through the apparatus.

The conductive tabs may be arranged evenly around the outlet aperture 217. For example there may be two opposed conductive tabs, or four conductive tabs arranged at 90° intervals around the outlet aperture. The conductive tabs provide locations in which arcing preferentially occurs between conductive elements connected to the inner conductor and outer conductor of the coaxial transmission line. That is, arcing and hence plasma generation, occurs preferentially between the first electrode 218 and the second electrode 221. The relative dimensions of the first electrode 218 and second electrode 221 are selected in conjunction with the power supplied to the plasma generating region in order to achieve an electric field strength to strike and sustain the plasma. Where the gas is argon, the field strength required for breakdown may be 600 Vmm⁻¹, for example. For example, the first electrode 218 may have an outer diameter of 0.5 mm, and the second electrodes 221 may be radially spaced from the first electrode 218 by a distance equal to or less than 1 mm.

The insulating pipe 214 covers the side surface of the outlet aperture 217 beyond the plasma generating zone 205 to avoid unwanted arcing in locations away from the first and second electrode.

The plasma may be naturally directed out of a distal end of the insulating pipe 214 by the direction of the gas flow from the gas input pipe 211.

Meanwhile, the hollow inner conductor 202 conveys dry steam via the steam conduit 206 to the longitudinal passage in the first electrode 218 and onwards into the plasma generating zone 205, which may be referred to as a hydroxyl radical generating region. Here the plasma ionises the steam molecules to product hydroxyl radicals, which then flow out of the sterilisation device 200. The insulating pipe 214 may have an inner diameter selected to narrow the outlet aperture 217 in a manner increases the speed of gas as it exits the sterilisation device. This may aid dispersal of hydroxyl radicals over a region to be sterilised. For example, the insulating pipe 214 may have an outer diameter of 10 mm and an inner diameter of 8 mm.

As explained above, in one example, the first electrode 218 is itself a hollow pipe that forms a distal portion of the steam conduit 206.

However, in another example, the sterilisation device 200 may be operated by delivering a mixture of a gas and dry steam through the inlet 211 and through the fluid conduit 208. The inner conductor 202 and first electrode 218 need not be hollow in this arrangement. When operating in this way, a plasma may be generated at the plasma generating zone 205, or hydroxyl radical generating region, to ionise steam molecules and provide hydroxyl radicals in substantially the same manner as described above.

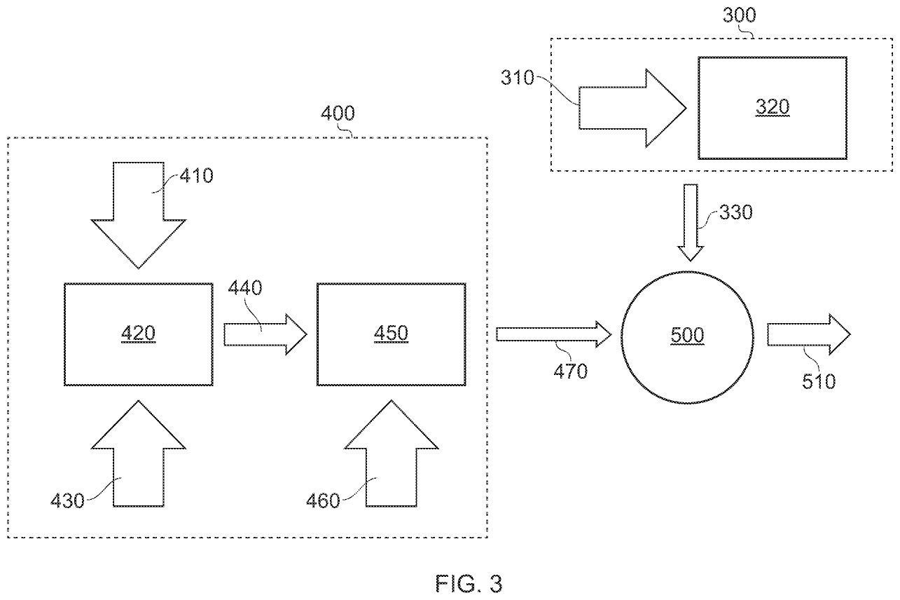

FIG. 3 shows a schematic diagram of a gas supply 300 and a steam supply 400 which may be used in an embodiment of the present invention to supply gas and dry steam to a sterilisation apparatus. The gas supply 300 is configured to deliver heated gas from an output 330 and into a mixer 500. The steam supply 400 is configured to deliver superheated steam from an output 470 and into the mixer 500. The mixer 500 is configured to receive the heated gas and the superheated steam and deliver a mixture of the gas and steam through a mixer output 510 to a sterilisation device, for example the sterilisation device described above with respect to FIG. 2. For example, the output gas and steam mixture may comprise less than 30% by volume of steam. For example, the flow rate of steam may be no than 4 litres per minute and the flow rate of gas may be at least 10 litres per minute.

In some examples, the mixer 500 may be heated to ensure that the gas and dry steam mixture is received by the sterilisation device at a high temperature to reduce or eliminate steam condensing within the sterilisation device and thereby ensure efficient generation of hydroxyl radicals. In some examples, the mixer 500 may be a venturi mixer which is configured to receive independent streams of gas from the gas supply 300 and steam from the steam supply 400, and output a mixed stream at an output 510. By using a venturi mixer in this way, the flow rate of the combined gas and steam may be increased, which can increase the rate of radical production by the sterilisation device and may also aid the projection of hydroxyl radicals from the outlet of the sterilisation device and towards an object or area to be sterilised.

In the arrangement shown, the gas supply 300 is a source of heated gas for delivery to the sterilisation device, where it is used to generate a plasma which ionises steam to produce hydroxyl radicals in the manner described above. The gas supply 300 comprises a source 310 of an inert gas, preferably Argon gas, which is connected to deliver the inert gas to a gas heater 320. The gas supply 310 may be a pressurised container, for example, and may comprise a controllable valve which allows a user to moderate the flow of gas from the gas supply 310. For example, the gas supply 310 may be arranged to supply gas at a rate of between 2 and 6 litres per minute. The gas heater 320 may comprise a heating element which is positioned in a gas flow path between the gas source 310 and the sterilisation device, in some examples. The gas heater 320 may be a heating device as described below with respect to FIG. 4 or FIG. 5. The gas heater 320 is configured to heat the inert gas to a temperature of at least 100° C. before the gas flows into the mixer 500 via an output 330 of the gas supply 300. The output 330 may comprise a valve to allow control over the flow rate of heated gas which is delivered to the mixer 500, for example between 2 and 6 litres of gas per minute.

The steam supply 400 is arranged to receive water at an input 410, and to deliver dry steam to the mixer 500 through the output 470. The steam supply 400 comprises a steam generator 420 and a steam dryer 450. Preferably, the steam supply 400 is arranged to deliver dry steam at a rate of between 2 and 6 litres of dry steam per minute. For example, the gas and dry steam flow rates may be matched, or may be modified in order to change the gas and dry steam composition delivered to the sterilisation device.

The steam generator 420 operates as a water boiler to produce wet steam (unsaturated steam, that is steam having entrained water droplets or water vapour). The steam generator 420 receives water 410 and applies heat (indicated by arrow 430) to generate steam. For example, heat may be applied to water using a resistive heating element. The flow rate of water into the steam generator 420 may depend on the flow rate of dry steam which is desired. In a preferred embodiment, water may be delivered to the steam generator 420 at a rate of around 4 litres per minute. The steam generator 420 preferably receives a steady supply of water during operation such that the steam generator 420 provides a constant supply of wet steam to the steam dryer 450. The supply of water may be controlled in order to control the amount of steam which is produced by the steam generator 420 and passed to the steam dryer 450. Steam passing from the steam generator 420 to the steam dryer 450 is indicated by arrow 440.

The steam dryer 450 is arranged to receive wet steam from the steam generator 420 and provide dry steam (that is, steam having no entrained water droplets or water vapour) to the mixer 500 through the output 470. In preferred embodiments, the steam dryer 450 is configured to superheat the steam, but it is also envisaged that the steam dryer 450 may comprise a steam trap, gauze, labyrinth or the like which is suitable for removing water vapour from a flow of wet steam provided from the steam generator 420 without the application of heat.

The steam dryer 450 receives wet steam (indicated by arrow 440) and applies heat (indicated by arrow 460) in order to superheat the steam to a temperature of at least 100° C., preferably at least 130° C. For example, the heat may be applied using a resistive heating element or in any other suitable manner. In some examples, the heating element may be arranged to heat a portion of a steam flow path between the steam generator and the sterilisation device, either directly or indirectly. For example, the steam dryer 450 may comprise a heating element which is positioned in the steam flow path between the steam generator 420 and the sterilisation device.

By superheating wet steam from the steam generator 420, all entrained water droplets or water vapour may be boiled to produce a flow of dry steam which is passed through the outlet 470 and to the mixer 500 to be combined with gas from the gas supply 300 and delivered to the sterilisation device through mixer output 510. For example, the steam dyer 450 may be a heating device such as described below with respect to FIG. 4 or FIG. 5.

FIG. 4 shows a cross-section view of a first heating device 600 which may be used in embodiments of the present invention. The heating device 600 may be used to heat a fluid which flows therethrough, and in particular may be used to heat gas, for example steam or an inert gas such as Argon. The heating device 600 may be used as a steam dryer or superheater (for example steam dryer 450 as shown in FIG. 3) and/or as a gas heater (for example gas heater 320 as shown in FIG. 3). In some examples, the heating device 600 may be used to heat a combined water mist and gas stream, in order to boil the water mist to form a dry steam and gas stream which can be delivered to the sterilisation device.

The heating device 600 comprises a heating element 610 which is positioned in a fluid flow path 620 such that the heating element 610 is arranged to heat the fluid which flows along the flow path 620. The flow path 620 is contained within a pipe 630, which forms a body of the heating device 600, with the heating element 610 being arranged generally along a longitudinal axis of the pipe. Fluid flows into the pipe 630 at an inlet 640 and leaves the pipe at an outlet 650. In some embodiments, the pipe 630 may be heated, either in addition to or as an alternative to the heating element 610, in order to aid heating of fluid passing through the pipe 630. In some embodiments, the pipe 630 may be partially or entirely contained within a housing to ensure that it cannot be touched by a user when the heating device 600 is in use.

The heating element 610 is preferably a resistive heating element, and so comprises a cable 615 for connection to a power source, such as a battery or mains power. For example, the heating element 610 may have a rating of around 70 W or more, and may be configured such that in use it may reach a temperature of at least 100° C., preferably 130° C. or more, in order to heat fluid which passes along the flow path 620 and past the heating element 610.

The fluid flow path 620 may be a flow path for steam, for example, or of an inert gas such as Argon gas. In some embodiments of the present invention, a heating device 600 is positioned in a flow path of steam between a steam generator and a sterilisation device in order to superheat the steam. A heating device 600 may additionally or alternatively be positioned in a flow path of gas between a gas source and a sterilisation device in order to heat the gas which is provided to the sterilisation device.

Although not shown in FIG. 4, in some embodiments the heating device 600 may comprise a thermometer which is configured to measure the temperature of fluid in the pipe 630 and/or measure the temperature of the heating element 610. This may be used to control the heating element 610 to achieve the desired fluid temperature. For example, the thermometer may extend into the fluid flow path 620 in order to determine if steam or gas passing along the fluid flow path 620 is heated to a suitable temperature. In some examples the flow rate of dry steam may be adjusted by changing the temperature of superheated steam, and so a thermometer may be useful in order to allow a user to monitor and control the flow rate of dry steam.

FIG. 5 shows a cross-section view of a second heating device 700 which may be used in embodiments of the present invention. The heating device 700 is used to heat a fluid which flows therethrough, and in particular may be used to heat gas, for example steam or an inert gas such as Argon. The heating device 700 may be used as a steam dryer or superheater (for example steam dryer 450 as shown in FIG. 3) and/or as a gas heater (for example gas heater 320 as shown in FIG. 3). In some examples, the heating device 700 may be used to heat a combined water mist and gas stream, in order to boil the water mist to form a dry steam and gas stream which can be delivered to the sterilisation device.

The heating device comprises a heating element 710, which is preferably a resistive heating element and so comprises a cable 715 for connection to a power source, such as a battery or mains power. For example, the heating element may have a rating of at least 1000 W, such as 1500 W or more, and may be configured such that in use it may reach a temperature of at least 100° C., preferably at least 140° C. or more, such as 200° C.

A pipe 720 is provided around the heating element 710 in a spiral or helix configuration. The pipe 720 provides a fluid flow path proximate to the heating element 710 such that the heating element 710 is arranged to heat the pipe 720, thereby heating fluid flowing along the flow path. Fluid flows into the pipe 720 at an inlet 730 and flows out of the pipe 720 at an outlet 740. Where the heating device 700 is used to heat steam, the pipe 720 provides a portion of a steam flow path between the steam generator and the sterilisation apparatus. Where the heating device 700 is used to heat an inert gas, such as Argon gas, the pipe 720 provides a portion of a gas flow path between a gas source and the sterilisation device.

To provide consistent and controlled heating of the pipe 720 and the fluid flowing therethrough, the pipe 720 is immersed in a reservoir 750 of a liquid having a high specific heat capacity. Although the pipe 720 is shown in a spiral or helix configuration, which increases the length of flow path of fluid proximate to the heating element 710 and within the reservoir 750 which ensures the fluid is heated to the desired temperature, it will be appreciated that the pipe 720 may follow any suitable path. The diameter of the pipe 720 affects the surface area of fluid being heated to the liquid within the reservoir 750. Preferably the diameter of the pipe 720 may be 5 mm or 6 mm, but any suitable diameter may be used to achieve the desired heating of fluid flowing through the pipe 720.

As shown in FIG. 5, the heating element 710 is also immersed in the reservoir 750, though it is envisaged that in some arrangements the heating element 710 may be positioned externally of the reservoir 750 to heat the liquid therein. For example, the liquid having a high specific heat capacity may be an oil, such as silicone oil or the like. By passing fluid through the pipe 720 immersed in a reservoir 750 of heated liquid in this way, the heating device 700 provides an efficient way to heat fluid (e.g. an inert gas, or steam) to a desired temperature. For example, the heating element may be arranged to heat the liquid within the reservoir 750 to a temperature of at least 140° C., for example 180° C., or 200° C. Such temperatures may be particularly suitable for ensuring that steam passing through the pipe from a wet steam generator is superheated when it reaches the outlet 740.

A thermometer 760 is provided to measure the temperature of the liquid within the reservoir 750 and may be used to control the heating element 710 to keep the liquid at a suitable temperature for heating fluid within the pipe 720 to a desired temperature. In some examples the flow rate of dry steam may be adjusted by changing the temperature of superheated steam, and so a thermometer may be useful in order to allow a user to monitor and control the flow rate of dry steam.

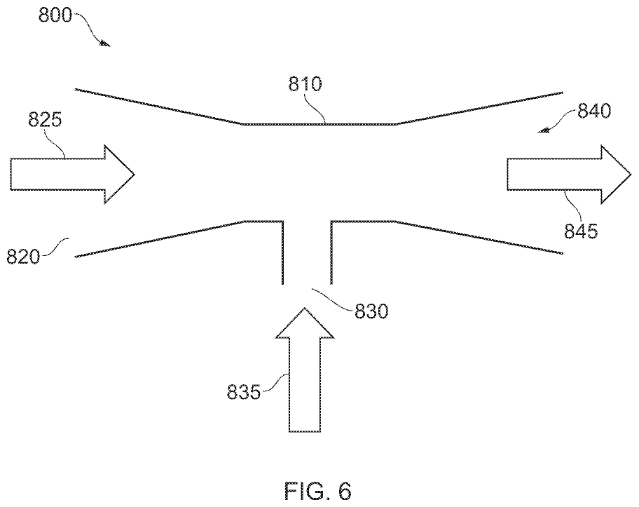

FIG. 6 shows a cross-section view of a venturi mixer 800 which may be used with embodiments of the present invention. For example, the venturi mixer 800 may be used as a mixer 500 as described above with respect to FIG. 3.

The venturi mixer 800 has a generally tubular housing 810, which is flared towards each end and which is tapered at its central section. A first end of the device forms a first inlet 820 into which dry or superheated steam may be delivered (indicated by arrow 825). A second inlet 830 is provided at the tapered central section of the housing 810, and may be used as an inlet for heated gas (indicated by arrow 835) for example. A second end of the device forms an outlet 840 from which the combined steam and gas stream flows towards the sterilisation device. For example, the output gas and steam mixture may comprise less than 30% by volume of steam. For example, the flow rate of steam may be no than 4 litres per minute and the flow rate of gas may be at least 10 litres per minute.

By using a venturi mixer 800, the flow rate of the combined gas and steam may be increased, which can increase the rate of radical production by the sterilisation device and may also aid the projection of hydroxyl radicals from the outlet of the sterilisation device and towards an object or area to be sterilised. In some embodiments the venturi mixer 800 may be heated to ensure that the temperature of gas and/or steam does not drop before being introduced to the sterilisation device.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A sterilisation apparatus comprising:
a sterilisation device having a hydroxyl radical generating region and an outlet for directing generated hydroxyl radicals out of the hydroxyl radical generating region towards a region to be sterilised;
a steam supply connected to deliver dry steam to the sterilisation device; and
a gas supply connected to supply gas to the sterilisation device;
wherein the sterilisation device is configured to generate a high impedance at the hydroxyl radical generating region when dry steam, radiofrequency (RF) or microwave frequency electromagnetic (EM) energy, and gas are delivered to the hydroxyl generating region, thereby to create a plasma of the gas which forms an ionisation discharge for generating hydroxyl radicals from the dry steam for delivery out of the hydroxyl generating region; and
wherein the gas supply comprises a gas heating element such that the gas supply is a source of heated gas.

2. A sterilisation apparatus according to claim 1, wherein the steam supply comprises a water mist generator and a heating element, wherein the heating element is arranged to receive water mist from the water mist generator and heat the mist to produce dry steam.

3. A sterilisation apparatus according to claim 2, further comprising a gas supply connected to supply gas to the sterilisation device, wherein gas from the gas supply is delivered to the mist generator in order to entrain mist in a flow of the gas.

4. A sterilisation apparatus according to claim 2, wherein the heating element is positioned in a mist flow path between the water mist generator and the sterilisation device.

5. A sterilisation apparatus according to claim 2, wherein at least a portion of a mist flow path between the water mist generator and the sterilisation device is provided by a pipe, wherein the heating element is arranged to heat the pipe.

6. A sterilisation apparatus according to claim 5, wherein the pipe is immersed in a reservoir of a liquid having a high specific heat capacity, and wherein the heating element is arranged to heat the liquid within the reservoir.

7. A sterilisation apparatus according to claim 1, wherein the steam supply comprises a steam generator and a steam dryer, wherein the steam dryer is arranged to receive wet steam from the steam generator and remove water droplets from the wet steam to produce dry steam.

8. A sterilisation apparatus according to claim 7, wherein the steam dryer comprises a steam heating element, and wherein the steam dryer is configured to heat the wet steam to produce superheated steam.

9. A sterilisation apparatus according to claim 8, wherein the steam heating element is positioned in a steam flow path between the steam generator and the sterilisation device.

10. A sterilisation apparatus according to claim 8, wherein the steam dryer comprises a pipe which forms at least a portion of a steam flow path between the steam generator and the sterilisation device, wherein the steam heating element is arranged to heat the pipe.

11. A sterilisation apparatus according to claim 10, wherein the pipe is immersed in a reservoir of a liquid having a high specific heat capacity, and wherein the steam heating element is arranged to heat the liquid within the reservoir.

12. (canceled)

13. A sterilisation apparatus according to claim 1, wherein the gas heating element is positioned in a gas flow path between the gas supply and the sterilisation device.

14. A sterilisation apparatus according to claim 1, wherein at least a portion of a gas flow path between the gas supply and the sterilisation device is provided by a pipe, wherein the gas heating element is arranged to heat the pipe.

15. A sterilisation apparatus according to claim 14, wherein the pipe is immersed in a reservoir of a liquid having a high specific heat capacity, and wherein the gas heating element is arranged to heat the liquid within the reservoir.

16. A sterilisation apparatus according to claim 1, wherein the gas supply is configured to heat the gas to a temperature of at least 100° C.

17. A sterilisation apparatus according to claim 1, further comprising a heater for heating the sterilisation device.

18. A sterilisation apparatus according to claim 1, further comprising a mixer arranged to receive dry steam from the steam supply and gas from the gas supply and deliver a dry steam and gas mixture to the sterilisation device, wherein the mixer is heated.

19. A sterilisation apparatus according to claim 1, wherein the gas supply is a supply of Argon gas.
